SEQUENCE LISTING (6 sequences)

SEQ ID NO: 1 - DNA, 26 bp, Chikungunya virus
  prim_transcript (1)..(26)
ttcgatgccatcatagccgcacactt

SEQ ID NO: 2 - DNA, 20 bp, Chikungunya virus
  prim_transcript (1)..(20)
ccctgctcccagcccccttg

SEQ ID NO: 3 - DNA, 28 bp, Chikungunya virus
  primer_bind (1)..(28)
cctcctacccaatgtacatacactattt

SEQ ID NO: 4 - DNA, 21 bp, Chikungunya virus
  primer_bind (1)..(21)
cctgttggcaaataccacgtt

SEQ ID NO: 5 - DNA, 24 bp, Chikungunya virus
  primer_bind (1)..(24)
aggaggctatgtccgtttctaaaa

SEQ ID NO: 6 - DNA, 22 bp, Chikungunya virus
  primer_bind (1)..(22)
tcatgacgttgtcctcaagcat

(19) United States
(12) Patent Application Publication
(10) Pub. No.: US 2012/0045761 A1
(43) Pub. Date: Feb. 23, 2012

(54) PROBES AND PRIMERS FOR DETECTION OF CHIKUNGUNYA

(75) Inventors: Manjula Jagannath, Karnataka (IN); Chandrasekhar Bhaskaran Nair, Karnataka (IN); Pillarisetti Venkata Subbarao, Karnataka (IN)

(73) Assignee: BIGTEC PRIVATE LIMITED, Bangalore, Karnataka (IN)

(21) Appl. No.: 13/203,286

(22) PCT Filed: Feb. 23, 2010
(86) PCT No.: PCT/IN2010/000103

§ 371 Date: Aug. 25, 2011

(30) Foreign Application Priority Data
Feb. 25, 2009 (IN) 00439/CHE/2009

(51) Int. Cl. C12Q 1/68

(52) U.S. Cl. 435/6.11

(57) ABSTRACT

The present disclosure gives a detailed description of methods for determining the presence of Chikungunya viral nucleic acids in blood/serum/plasma samples by employing “Oligonucleotide” probes. The designed “Oligonucleotide” probes can be used for qualitative or quantitative detection of Chikungunya virus in an infected sample by employing Real time PCR.

### TECHNICAL FIELD

The present disclosure is in relation to a method for the detection of Chikungunya viral infection using nucleic acids isolated from blood samples by employing “Oligonucleotide” probes. The method employed here for detection is by Real time PCR.

### BACKGROUND OF THE DISCLOSURE

Chikungunya virus is indigenous to tropical Africa and Asia, where it is transmitted to humans by the bite of infected mosquitoes, usually of the genus Aedes. Chikungunya virus belongs to alpha-virus under Toga virdae family. It is an “Arbovirus” (Ar-arthropod, bo-borne). CHIK fever epidemics are sustained by human-mosquito-human transmission. The word “Chikungunya” is thought to derive from description in local dialect of the contorted posture of patients afflicted with the severe joint pain associated with this disease. The main virus reservoirs are monkeys, but other species can also be affected, including humans.

Chikungunya (in the Makonde language “that which bends up”) virus (CHIKV) is an insect-borne virus, of the genus, Alphavirus that is transmitted to humans by virus-carrying Aedes mosquitoes. There have been recent outbreaks of CHIKV associated with severe morbidity. CHIKV causes an illness with symptoms similar to dengue fever. CHIKV manifests itself with an acute febrile phase of the illness that lasts only two to five days, followed by a prolonged arthralgic disease that affects the joints of the extremities. The pain associated with CHIKV infection of the joints persists for weeks or months.

The incubation period of Chikungunya disease is from two to four days. Symptoms of the disease include a fever up to 40° C. (104° F.), a petechial or maculopapular rash of the trunk and occasionally the limbs, and arthralgia or arthritis affecting multiple joints. Other nonspecific symptoms can include headache, conjunctival infection, and slight photophobia. Typically, the fever lasts for two days and then ends abruptly. However, other symptoms, namely joint pain, intense headache, insomnia and an extreme degree of prostration last for a variable period; usually for about 5 to 7 days. Patients have complained of joint pains for much longer time periods depending on their age. Common laboratory tests for Chikungunya include RT-PCR, virus isolation, and serological tests. Virus isolation provides the most definitive diagnosis but takes 1-2 weeks for completion and must be carried out in biosafety level 3 laboratories. The technique involves exposing specific cell lines to samples from whole blood and identifying Chikungunya virus-specific responses. RT-PCR using nested primer pairs to amplify several Chikungunya-specific genes from whole blood. Results can be determined in 1-2 days. Serological diagnosis requires a larger amount of blood than the other methods and uses an ELISA assay to measure Chikungunya-specific IgM levels. Results require 2-3 days and false positives can occur with infection via other related viruses such as O′ nyong'nyong virus and Semliki Forest Virus.

### STATEMENT OF THE DISCLOSURE

Accordingly, the present disclosure relates to probes having SEQ ID Nos. 1 and 2; probes having SEQ ID Nos. 1 and 2 conjugated with detectable labels at 5′ end or 3′ end or both; primers of SEQ ID Nos. 3, 4, 5 and 6; a PCR reaction mixture for detection of chikungunya, said mixture comprising the sample to be detected, nucleic acid amplification reagents, probes selected from a group comprising SEQ ID Nos. 1 and 2, and corresponding primers selected from a group comprising SEQ ID Nos. 3, 4, 5 and 6; a method of detecting and optionally quantifying chikungunya infection, said method comprising steps of—a) forming a reaction mixture comprising a sample to be detected, nucleic acid amplification reagents, probes selected from a group comprising SEQ ID Nos. 1 and 2 and corresponding primers selected from a group comprising SEQ ID Nos. 3, 4, 5 and 6, b) subjecting the reaction mixture to PCR to obtain copies of target sequence followed by measuring any increase in fluorescence signal for detecting the chikungunya infection and c) optionally constructing a standard curve from the detected signal to obtain copy number for quantifying the chikungunya infection; and a kit for detection of chikungunya infection, said kit comprising probes of SEQ ID Nos. 1 and 2, individually or in combination; corresponding pair of primers of SEQ ID Nos. 3, 4, 5 and 6, individually or in combination and amplification reagent.

### BRIEF DESCRIPTION OF ACCOMPANYING FIGURE

FIG. 1 shows Chikungunya standard curve.

### DETAILED DESCRIPTION OF THE DISCLOSURE

The present disclosure relates to probes having SEQ ID Nos. 1 and 2.

In an embodiment of the present disclosure, said probes are for detection of chikungunya.

The present disclosure relates to probes having SEQ ID Nos. 1 and 2 conjugated with detectable labels at 5′ end or 3′ end or both.

In an embodiment of the present disclosure the probes are conjugated with fluorophore at the 5′ end and quencher at the 3′ end.

In another embodiment of the present disclosure said fluorophore is selected from a group comprising fluorescein and fluorescein derivatives VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein (FAM), tetrachloro-6-carboxyfluoroscein, 5-carboxyrhodamine and cyanine dyes.

In yet another embodiment of the present disclosure said quencher is selected from a group comprising Tetra Methyl Rhodamine, 4′-(4-dimethylaminophenylazo)benzoic acid, 4-dimethylaminophenylazophenyl-4′-maleimide,tetramethylrhodamine, carboxytetramethylrhodamine and Black Hole Quencher dyes.

In still another embodiment of the present disclosure the preferred Fluorophore is 6-Carboxy Fluorescein [FAM] at 5′ end and the preferred quencher is Tetra Methyl Rhodamine [TAMRA] at 3′ end.

The present disclosure is in relation to primers of SEQ ID Nos. 3, 4, 5 and 6.

In an embodiment of the present disclosure the primers having SEQ ID Nos 3 and 4 are sense primers and the primers having SEQ ID Nos 5 and 6 are anti-sense primers.

In another embodiment of the present disclosure the primers having SEQ ID Nos 3 and 5 correspond to probe of SEQ ID No. 1 and the primers having SEQ ID Nos 4 and 6 correspond to probe of SEQ ID No. 2.

In yet another embodiment of the present disclosure the probes having SEQ ID Nos. 1 and 2 are optionally conjugated with detectable labels at 5′ end or 3′ end or both.

The present disclosure relates to a PCR reaction mixture for detection of chikungunya, said mixture comprising the sample to be detected, nucleic acid amplification reagents, probes selected from a group comprising SEQ ID Nos. 1 and 2, and corresponding primers selected from a group comprising SEQ ID Nos. 3, 4, 5 and 6.

In an embodiment of the present disclosure the primers having SEQ ID Nos 3 and 5 correspond to the probe of SEQ ID No. 1 and the primers having SEQ ID Nos 4 and 6 correspond to the probe of SEQ ID No. 2.

In another embodiment of the present disclosure the probes having SEQ ID Nos. 1 and 2 are optionally conjugated with detectable labels at 5′ end or 3′ end or both.

In yet another embodiment of the present disclosure the sample is selected from a group comprising blood, serum and plasma.

The present disclosure relates to a method of detecting and optionally quantifying chikungunya infection, said method comprising steps of:
a) forming a reaction mixture comprising a sample to be detected, nucleic acid amplification reagents, probes selected from a group comprising SEQ ID Nos. 1 and 2 and corresponding primers selected from a group comprising SEQ ID Nos. 3, 4, 5 and 6;
b) subjecting the reaction mixture to PCR to obtain copies of target sequence followed by measuring any increase in fluorescence signal for detecting the chikungunya infection; and
c) optionally constructing a standard curve from the detected signal to obtain copy number for quantifying the chikungunya infection.

In an embodiment of the present disclosure the primers having SEQ ID Nos 3 and 4 are sense primers and the primers having SEQ ID Nos 5 and 6 are anti-sense primers and wherein the sample is selected from a group comprising blood, serum and plasma.

In another embodiment of the present disclosure the primers having SEQ ID Nos 3 and 5 correspond to the probe of SEQ ID No. 1 and the primers having SEQ ID Nos 4 and 6 correspond to the probe of SEQ ID No. 2.

In yet another embodiment of the present disclosure the probes having SEQ ID Nos. 1 and 2 are conjugated with detectable labels at 5′ end or 3′ end or both and wherein the fluorescence signal is generated by the probes having fluorophore at the 5′ end along with the quencher at 3′ end.

In still another embodiment of the present disclosure the fluorophore is selected from a group comprising fluorescein and fluorescein derivatives VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein (FAM), tetrachloro-6-carboxyfluoroscein, 5-carboxyrhodamine and cyanine dyes and wherein the quencher is selected from a group comprising Tetra Methyl Rhodamine, 4′-(4-dimethylaminophenylazo)benzoic acid, 4-dimethylaminophenylazophenyl-4′-maleimide,tetramethylrhodamine, carboxytetramethylrhodamine and Black Hole Quencher dyes.

The present disclosure relates to a kit for detection of chikungunya infection, said kit comprising probes of SEQ ID Nos. 1 and 2, individually or in combination; corresponding pair of primers of SEQ ID Nos. 3, 4, 5 and 6, individually or in combination and amplification reagent.

In an embodiment of the present disclosure probes having SEQ ID Nos. 1 and 2 are optionally conjugated with detectable labels at 5′ end or 3′ end or both.

In another embodiment of the present disclosure said amplification reagent is a combination comprising magnesium chloride, Taq polymerase and buffer for amplification.

The principle objective of the present disclosure is the detection of Chikungunya viral infection using nucleic acids isolated from infected blood samples. The mode of detection is by monitoring increase in fluorescence by employing real time PCR using “Oligonucleotide” probes labeled with a fluorophore and a quencher.

### Probe and Primer Designing

Probe having SEQ ID No. 1 along with primers having SEQ ID Nos. 3 and 5 were designed for the Nonstructural protein nsP4 gene of Chikungunya. Similarly SEQ ID No. 2 probe along with SEQ ID Nos. 4 and 6 primers were designed for the Structural protein gene of Chikungunya.

The present disclosure is in relation to “Oligonucleotide” probes designated as SEQ ID No. 1 probe along with primers designated as SEQ ID Nos. 3 and 5 for the detection of Chikungunya viral infection, wherein said primers are sense and anti-sense primers respectively and SEQ ID No. 2 probe along with primers designated as SEQ ID Nos. 4 and 6 for the detection of Chikungunya viral infection wherein said primers are sense and anti-sense primers respectively.

According to the present disclosure SEQ ID No. 1 probe along with its respective sense and anti-sense primers is designed for the Nonstructural protein nsP4 gene of Chikungunya. Similarly SEQ ID No. 2 probe along with its corresponding sense and anti-sense primers is designed for Structural protein gene of Chikungunya. According to the present disclosure said “Oligonucleotide” probes are conjugated to detectable labels having fluorophore at 5′ end and quencher at the 3 ‘end. The fluorophore is selected from a group comprising fluorescein and fluorescein derivatives FAM, VIC, JOE, 5-(2’-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes.

In still another embodiment of the present disclosure said quencher is selected from a group comprising Tetra Methyl Rhodamine, 4′-(4-dimethylaminophenylazo) benzoic acid, 4-dimethylaminophenylazophenyl-4′-maleimide, tetramethylrhodamine, carboxytetramethylrhodamine and BHQ dyes.

In yet another embodiment of the present disclosure said fluorophore is 6-Carboxy Fluorescein [FAM] and the quencher is Tetra Methyl Rhodamine [TAMRA].

In still another embodiment of the present disclosure said detection is qualitative or quantitative in nature.

The present disclosure is in relation to a PCR reaction mixture for the detection of Chikungunya viral infection, wherein said mixture comprises of nucleic acid amplification reagents, “Oligonucleotide” probes designated as SEQ ID No. 1 or SEQ ID No. 2 in combination with primers designated as SEQ ID Nos. 3 and 5 or SEQ ID Nos. 4 and 6 and Chikungunya nucleic acid isolated from blood/serum/plasma samples. The present disclosure is in relation to a method for detecting Chikungunya viral infection, where in the said PCR mixture comprising of nucleic acid amplification reagents, “Oligonucleotide” probes designated as SEQ ID No. 1 or SEQ ID No. 2 along with their corresponding primers and a test sample is subjected for amplification using real-time PCR to obtain copies of the target sequence. The amplification is measured in terms of increase in fluorescence signal.

The “Oligonucleotide” probe has a size ranging from 20-26 nucleotides. The designed probe has a fluorophore at the 5′ end and quencher at the 3′ end. The fluorophore at the 5′ end is 6-Carboxy Fluorescein [FAM] and the quencher is Tetra Methyl Rhodamine [TAMRA] when present at the 3′ end. The current disclosure is used for the detection of Chikungunya viral infection present in blood/serum/plasma samples. The method used for detection is by monitoring the increase in fluorescence during the PCR.

According to the present disclosure the “Oligonucleotide probe” refers to a short sequence of deoxyribonucleic acid (DNA). The Oligonucleotide probe can specifically hybridise to the target DNA without exhibiting non-specific hydridisation to uninfected DNA.

The probes employed here follow the principles of Taqman chemistry. TaqMan probes also called Double-Dye oligonucleotide or dual labeled probes, are the most widely used type of probes.

The “Oligonucleotide” probe according to the present invention, therefore, is further provided in combination with their corresponding sense and anti-sense primers that can be used to specifically amplify and detect Chikungunya viral sequences in a test sample by real time PCR. One can also quantify the viral load based on the Ct obtained from a standard curve.

The probes having SEQ ID Nos. 1 and 2 along with their corresponding primers have sequences as described in Table 1 & Table 2.

TABLE. 1

| Sequence name | Nucleotide Sequence |
|---|---|
| SEQ ID No. 1 | 5′-TTCGATGCCATCATAGCCGCACACTT-3′ |
| SEQ ID No. 3 | 5′-CCTCCTACCCAATGTACATACACTATTT-3′ |
| SEQ ID No. 5 | 5′-AGGAGGCTATGTCCGTTTCTAAAA-3′ |

TABLE. 2

| Sequence name | Nucleotide Sequence |
|---|---|
| SEQ ID No. 2 | 5′-CCCTGCTCCCAGCCCCCTTG-3′ |
| SEQ ID No. 4 | 5′-CCTGTTGGCAAATACCACGTT-3′ |
| SEQ ID No. 6 | 5′-TCATGACGTTGTCCTCAAGCAT-3′ |

The SEQ ID No. 1 and 2 can be further conjugated with Fluorophore and Quencher as represented below:

5′-Flurophore-TTCGATGCCATCATAGCCGCACACTT-
Quencher-3′
5′-Flurophore-CCCTGCTCCCAGCCCCCTTG-Quencher-3′

The present disclosure is further elaborated by the following examples and figures. However, these examples should not be construed to limit the scope of the disclosure.

### Example 1

To gain a better understanding of the above invention, a study was done on established sample panels and infected samples collected from blood, serum and plasma with the probes of the present disclosure having SEQ ID Nos. 1 and 2. Positive results were obtained for the study and the results are highlighted as below:

A sample panel consisting of 10 Chikungunya positives and 10 Chikungunya negative samples were subjected to Real time PCR using probes having SEQ ID Nos. 1 and 2 along with their corresponding sense and anti-sense primers. The PCR mix composition and reactions conditions are as given in table 3 & 4. Amplification was measured in terms of increase in fluorescence signal during the course of the PCR reaction.

TABLE 3
Real time-PCR mix composition
Real time PCR mix Composition

| | | |
|---|---|---|
| Reverse transcriptase | 1 | μl (10 units) |
| Premix | 5.0 | μl |
| Forward Primer | 0.2 | μl (2 picomoles) |
| Reverse Primer | 0.2 | μl (2 picomoles) |
| Probe | 0.2 | μl (2 picomoles) |
| Sample | 2.0 | μl |
| Water | 1.4 | μl |
| Total | 10 | μl |

TABLE 4
Real time-PCR cycle conditions
PCR Program

| | |
|---|---|
| Step 1 (CDNA synthesis) | 48° C. for 10 min |
| Step 2 (initial denaturation) | 95° C. for 60 sec |
| Step 3 (cycle denaturation) | 95° C. for 5 sec |
| Step 4 (annealing & extension) | 60° C. for 34 sec |

Step 3 and 4 repeated 40 times

Results obtained showed that;

SEQ ID No. 1, the probe designed for the non-structural nsP4 gene picked up all the 10 predetermined positives within 40 cycles (positive sample cut off).

SEQ ID No. 2, the probe designed for structural gene picked up all the 10 predetermined positives within 40 cycles (positive sample cut off). They did not show any false amplification with the negative samples. Both the probes having SEQ ID Nos. 1 and 2 showed 100% sensitivity and specificity in picking all the positive samples. Please refer Table 5.

TABLE 5

| Sl. No | Sample ID | SEQ ID No. 1 Ct | SEQ ID No. 2 Ct |
|---|---|---|---|
| 1 | Positive 1 | 17.22 | 18.0776 |
| 2 | Positive 2 | 21.3024 | 20.019 |
| 3 | Positive 3 | 29.1473 | 27.2032 |
| 4 | Positive 4 | 15.5172 | 16.3176 |
| 5 | Positive 5 | 16.3803 | 16.2046 |
| 6 | Positive 6 | 17.632 | 17.883 |
| 7 | Positive 7 | 15.8448 | 17.096 |
| 8 | Positive 8 | 17.1559 | 13.8004 |
| 9 | Positive 9 | 14.3997 | 14.9991 |
| 10 | Positive 10 | 18.7158 | 14.7021 |

### Example 2

Further, it is also possible to quantify the parasite load from an infected sample collected from blood, serum or plasma, by comparing the Ct values obtained from a standard curve.

### Protocol for Calculation of Copy Number by Plaque Assay

Confluent monolayers of Vero was prepared in 6 well plates. 10-fold dilutions (10¹ to 10⁷) of virus was prepared in chilled maintenance medium (MEM, with 1% serum). The culture medium was then removed and 0.2 ml of the virus inoculums was then added starting from the highest dilution. Care was taken to ensure that a film of medium completely covered the cell sheet. The plate was then incubated at 37° C. for 1 hour with intermittent rocking of the plate. The inoculums were then removed with a pipette and 1.5 ml of agarose overlay medium (growth medium with 0.3% agarose and 2.5% FCS) was then added to it. Care was taken to ensure that the overlay medium was spread evenly over the monolayer, this was then left at room temperature for 10 mins and then incubated at 37° C. The monolayers were observed daily, starting from second day of incubation. Once the plaques developed usually by the fourth day post inoculation, the number of plaques at each dilution was then counted. The agarose overlay was removed and the monolayer was gently washed with PBS and the plate was stained with 0.1% crystal violet solution and the plaques were again counted. The virus titre was estimated as plaque forming units per ml (pfu/ml) by counting the number of plaques at appropriate dilution. For instance:—

Number of plaques produced=9
Dilution of virus=1×10⁵
Volume of inoculum=0.2 ml
Virus titre=9×1×10⁵×5 pfu per ml=4.5×10⁶

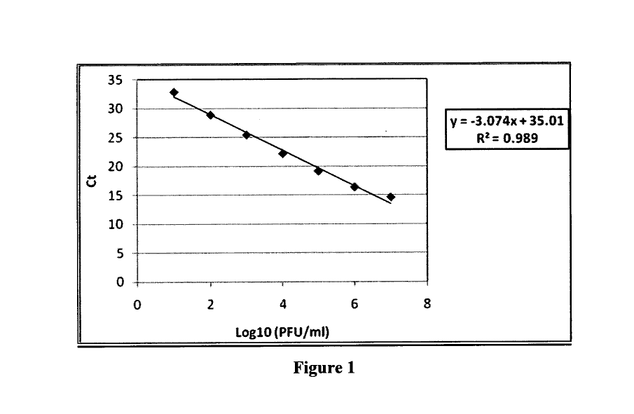

The standard curve is depicted in FIG. 1 and the standard curve values with respect to Ct are provided in Table 6.

TABLE 6
Standard curve values with respect to Ct

| log10 (PFU/ml) | Cycle number (Ct) |
|---|---|
| 1.00E+01 | 32.81 |
| 1.00E+02 | 28.79 |
| 1.00E+03 | 25.37 |
| 1.00E+04 | 22.13 |
| 1.00E+05 | 19.08 |
| 1.00E+06 | 16.31 |
| 1.00E+07 | 14.53 |

### CONCLUSION

a) Both the probes having SEQ ID Nos. 1 and 2 picked up all the positive samples. They did not show any false amplification with the negative samples. Thus showing 100% specificity and 100% sensitivity.

b) Based on the overall evaluation studies either of the probes having SEQ ID Nos. 1 and 2 can be used for Chikungunya detection based on real time PCR.

1) Probes having SEQ ID Nos. 1 and 2.

2) The probes as claimed in claim 1, wherein said probes are for detection of chikungunya.

3) Probes having SEQ ID Nos. 1 and 2 conjugated with detectable labels at 5′ end or 3′ end or both.

4) The probe as claimed in claim 3, wherein the probes are conjugated with fluorophore at the 5′ end and quencher at the 3′ end; and
wherein said fluorophore is selected from a group comprising fluorescein and fluorescein derivatives VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein (FAM), tetrachloro-6-carboxyfluoroscein, 5-carboxyrhodamine and cyanine dyes; and
said quencher is selected from a group comprising Tetra Methyl Rhodamine, 4′-(4-dimethylaminophenylazo)benzoic acid, 4-dimethylaminophenylazophenyl-4′-maleimide,tetramethylrhodamine, carboxytetramethylrhodamine and Black Hole Quencher dyes.

5) (canceled)

6) (canceled)

7) The probes as claimed in claim 4, wherein the fluorophore is 6-Carboxy Fluorescein [FAM] at 5′ end and the quencher is Tetra Methyl Rhodamine [TAMRA] at 3′ end.

8) Primers of SEQ ID Nos. 3, 4, 5 and 6.

9) The primers as claimed in claim 8, wherein the primers having SEQ ID Nos 3 and 4 are sense primers and the primers having SEQ ID Nos 5 and 6 are anti-sense primers; and
wherein the primers having SEQ ID Nos 3 and 5 correspond to probe of SEQ ID No. 1 and the primers having SEQ ID Nos 4 and 6 correspond to probe of SEQ ID No. 2; and
wherein the probes having SEQ ID Nos. 1 and 2 are optionally conjugated with detectable labels at 5′ end or 3′ end or both.

10) (canceled)

11) (canceled)

12) A PCR reaction mixture for detection of chikungunya, said mixture comprising the sample to be detected, nucleic acid amplification reagents, probes selected from a group comprising SEQ ID Nos. 1 and 2, and corresponding primers selected from a group comprising SEQ ID Nos. 3, 4, 5 and 6.

13) The reaction mixture as claimed in claim 12, wherein the primers having SEQ ID Nos 3 and 5 correspond to the probe of SEQ ID No. 1 and the primers having SEQ ID Nos 4 and 6 correspond to the probe of SEQ ID No. 2; and
wherein the probes having SEQ ID Nos. 1 and 2 are optionally conjugated with detectable labels at 5′ end or 3′ end or both.

14) (canceled)

15) The reaction mixture as claimed in claim 12, wherein the sample is selected from a group comprising blood, serum and plasma.

16) A method of detecting and optionally quantifying chikungunya infection, said method comprising steps of:
(a) forming a reaction mixture comprising a sample to be detected, nucleic acid amplification reagents, probes selected from a group comprising SEQ ID Nos. 1 and 2 and corresponding primers selected from a group comprising SEQ ID Nos. 3, 4, 5 and 6;
(b) subjecting the reaction mixture to PCR to obtain copies of target sequence followed by measuring any increase in fluorescence signal for detecting the chikungunya infection; and
(c) optionally constructing a standard curve from the detected signal to obtain copy number for quantifying the chikungunya infection.

17) The method as claimed in claim 16, wherein the primers having SEQ ID Nos 3 and 4 are sense primers and the primers having SEQ ID Nos 5 and 6 are anti-sense primers and wherein the sample is selected from a group comprising blood, serum and plasma.

18) The method as claimed in claim 17, wherein the primers having SEQ ID Nos 3 and 5 correspond to the probe of SEQ ID No. 1 and the primers having SEQ ID Nos 4 and 6 correspond to the probe of SEQ ID No. 2; and
wherein the probes having SEQ ID Nos. 1 and 2 are conjugated with detectable labels at 5′ end or 3′ end or both and wherein the fluorescence signal is generated by the probes having fluorophore at the 5′ end along with the quencher at 3′ end.

19) (canceled)

20) The method as claimed in claim 18, wherein the fluorophore is selected from a group comprising fluorescein and fluorescein derivatives VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein (FAM), tetrachloro-6-carboxyfluoroscein, 5-carboxyrhodamine and cyanine dyes and wherein the quencher is selected from a group comprising Tetra Methyl Rhodamine, 4′-(4-dimethylaminophenylazo)benzoic acid, 4-dimethylaminophenylazophenyl-4′-maleimide,tetramethylrhodamine, carboxytetrapemethylrhodamine and Black Hole Quencher dyes.

21) A kit for detection of chikungunya infection, said kit comprising probes of SEQ ID Nos. 1 and 2, individually or in combination; corresponding pair of primers of SEQ ID Nos. 3, 4, 5 and 6, individually or in combination and amplification reagent.

22) The kit as claimed in claim 21, wherein the probes having SEQ ID Nos. 1 and 2 are optionally conjugated with detectable labels at 5′ end or 3′ end or both.

23) The kit as claimed in claim 21, wherein said amplification reagent is a combination comprising magnesium chloride, Taq polymerase and buffer for amplification.
